(19) United States
(12) Patent Application Publication
(10) Pub. No.: US 2026/0269049 A1
(43) Pub. Date: Sep. 10, 2026

(54) MULTIMODAL SIGNAL PROCESSING FOR DIVERGENCE CHARACTERIZATION IN PERSONALIZED TREATMENT TAILORING

(71) Applicant: WONDER SCIENCES, INC., Marina del Rey, CA (US)

(72) Inventor: Ryan R. Magnussen, Marina del Rey, CA (US)

(21) Appl. No.: 19/559,223

(22) Filed: Mar. 6, 2026

Related U.S. Application Data
(60) Provisional application No. 63/768,827, filed on Mar. 7, 2025.

(51) Int. Cl. G16H 20/70

(52) U.S. Cl. CPC G16H 20/70

(57) ABSTRACT

A computer-implemented method detects cross-modal affective divergence in psychiatric assessment contexts by encoding multimodal biometric data into modality-specific affective state representations, projecting each representation into a shared affective latent space using contrastive alignment loss, computing pairwise divergence scores between aligned modality representations, generating a cross-modal affective divergence score representing clinical significance of detected divergence, computing per-modality trust weights indicating estimated reliability of each modality as an indicator of the patient’s affective state, producing a trust-weighted fused affective state estimate by combining aligned modality representations according to trust weights, and generating a personalized treatment plan based on the trust-weighted fused affective state estimate.

### CROSS-REFERENCE TO RELATED APPLICATIONS

The present application claims the benefit of and priority to U.S. Provisional Application No. 63/768,827 filed on March 7, 2025, which is incorporated by reference.

### BACKGROUND

Mental healthcare delivery faces substantial fragmentation across diagnostic, therapeutic, pharmacological, and administrative domains. Patients seeking mental health treatment encounter multiple disconnected systems, including separate electronic health record platforms, standalone diagnostic tools, isolated therapy management applications, and independent medication management systems. This fragmentation results in data silos, inconsistent treatment protocols, delayed interventions, and suboptimal patient outcomes.

Existing artificial intelligence applications in mental healthcare address individual aspects of this fragmentation but fail to provide integrated solutions. Prior art systems operate as standalone tools without integration into comprehensive clinical workflows, medication management, or longitudinal biometric tracking. Conventional AI systems suffer from significant privacy and security limitations through centralized data storage models that create single points of failure.

Current psychopharmacology management relies heavily on trial-and-error prescribing methodologies without integrating pharmacogenomic analysis, or real-time biometric feedback into unified optimization engines. The mental healthcare system faces a critical shortage of qualified providers, with demand for services far exceeding available clinical capacity, and existing AI tools do not adequately augment clinician capabilities in ways that meaningfully reduce clinical workload while maintaining or improving care quality.

Crisis prevention in mental healthcare remains largely reactive rather than predictive, with no existing systems integrating multimodal signals with longitudinal patient data, pharmacological context, and therapeutic history to provide continuous predictive crisis prevention within an integrated care ecosystem.

### SUMMARY

According to an aspect of the invention, a computer-implemented method detects cross-modal affective divergence in psychiatric assessment contexts by accessing multimodal biometric data from a patient captured by one or more sensors, encoding each modality of the multimodal biometric data into a modality-specific affective state representation by a plurality of modality-specific encoder neural networks, projecting each modality-specific affective state representation into a shared affective latent space by learned modality-specific alignment projections trained with a contrastive alignment loss, computing a pairwise divergence score between each pair of aligned modality representations in the shared affective latent space, computing a cross-modal affective divergence score representing the clinical significance of detected divergence between verbal expressions and non-verbal expressions, computing a per-modality trust weight indicating estimated reliability of each modality as an indicator of the patient’s affective state based at least in part on the cross-modal affective divergence score and producing a trust-weighted fused affective state estimate by combining the aligned modality representations according to the per-modality trust weights, and generating a personalized treatment plan based on the trust-weighted fused affective state estimate, providing a technical solution that preserves inter-modal disagreement as clinically informative signal rather than resolving it as noise.

Clause 1. A computer-implemented method for detecting cross-modal affective divergence in a psychiatric assessment context, comprising: accessing multimodal biometric data from a patient captured by one or more sensors; encoding, by a plurality of modality-specific encoder neural networks, each modality of the multimodal biometric data into a modality-specific affective state representation; projecting, by learned modality-specific alignment projections trained with a contrastive alignment loss, each modality-specific affective state representation into a shared affective latent space; computing a pairwise divergence score between each pair of aligned modality representations in the shared affective latent space; computing a cross-modal affective divergence score representing the clinical significance of detected divergence based on the pairwise divergence scores; computing, based at least in part on the cross-modal affective divergence score, a per-modality trust weight indicating estimated reliability of each modality as an indicator of the patient’s affective state; generating a trust-weighted fused affective state estimate by combining the aligned modality representations according to the per-modality trust weights; and generating a personalized treatment plan based on the trust-weighted fused affective state.

Clause 2. The computer-implemented method of clause 1, the multimodal biometric data comprising at least two selected from the group of: text data, voice audio data, facial imagery data, and physiological sensor data.

Clause 3. The computer-implemented method of clause 1, wherein each modality-specific encoder is independently trained.

Clause 4. The computer-implemented method of clause 1, wherein the contrastive alignment loss is configured to produce aligned representations that cluster together when modalities convey concordant affective states and separate when modalities convey discordant affective states.

Clause 5. The computer-implemented method of clause 1, wherein computing the pairwise divergence score comprises computing a complement of a cosine similarity between the pair of aligned modality representations in the shared latent space.

Clause 6. The computer-implemented method of clause 1, wherein computing the pairwise divergence score comprises: generating a divergence matrix comprising all pairwise divergence scores arranged in the divergence matrix.

Clause 7. The computer-implemented method of clause 1, further comprising: computing a verbal-non-verbal divergence score between a textual modality each non-textual modality; and identifying the maximum verbal-non-verbal divergence score, wherein computing the cross-modal affective divergence score is further based on the maximum verbal-non-verbal divergence score.

Clause 8. The computer-implemented method of clause 1, further comprising: computing a somatic coherence score representing similarity across non-verbal modalities, wherein computing the cross-modal affective divergence score is further based on the somatic coherence score.

Clause 9. The computer-implemented method of clause 8, wherein computing the somatic coherence score comprises: computing a cosine similarity between each pair of non-verbal modalities; and computing an average of the cosine similarities between the pairs of non-verbal modalities as the somatic coherence score.

Clause 10. The computer-implemented method of clause 1, further comprising: applying a divergence characterization model to the pairwise divergence scores and the cross-modal affective divergence score to output a classification of divergence pattern exhibited in the multimodal data, wherein generating the personalized treatment plan is further based on the classification of the divergence pattern.

Clause 11. A non-transitory computer-readable storage medium storing instructions for detecting cross-modal affective divergence in a psychiatric assessment context, the instructions that, when executed, cause one or more processors to perform operations comprising: accessing multimodal biometric data from a patient captured by one or more sensors; encoding, by a plurality of modality-specific encoder neural networks, each modality of the multimodal biometric data into a modality-specific affective state representation; projecting, by learned modality-specific alignment projections trained with a contrastive alignment loss, each modality-specific affective state representation into a shared affective latent space; computing a pairwise divergence score between each pair of aligned modality representations in the shared affective latent space; computing a cross-modal affective divergence score representing the clinical significance of detected divergence based on the pairwise divergence scores; computing, based at least in part on the cross-modal affective divergence score, a per-modality trust weight indicating estimated reliability of each modality as an indicator of the patient’s affective state; generating a trust-weighted fused affective state estimate by combining the aligned modality representations according to the per-modality trust weights; and generating a personalized treatment plan based on the trust-weighted fused affective state.

Clause 12. The non-transitory computer-readable storage medium of clause 11, the multimodal biometric data comprising at least two selected from the group of: text data, voice audio data, facial imagery data, and physiological sensor data.

Clause 13. The non-transitory computer-readable storage medium of clause 11, wherein each modality-specific encoder is independently trained.

Clause 14. The non-transitory computer-readable storage medium of clause 11, wherein the contrastive alignment loss is configured to produce aligned representations that cluster together when modalities convey concordant affective states and separate when modalities convey discordant affective states.

Clause 15. The non-transitory computer-readable storage medium of clause 11, wherein computing the pairwise divergence score comprises computing a complement of a cosine similarity between the pair of aligned modality representations in the shared latent space.

Clause 16. The non-transitory computer-readable storage medium of clause 11, wherein computing the pairwise divergence score comprises: generating a divergence matrix comprising all pairwise divergence scores arranged in the divergence matrix.

Clause 17. The non-transitory computer-readable storage medium of clause 11, the operations further comprising: computing a verbal-non-verbal divergence score between a textual modality each non-textual modality; and identifying the maximum verbal-non-verbal divergence score, wherein computing the cross-modal affective divergence score is further based on the maximum verbal-non-verbal divergence score.

Clause 18. The non-transitory computer-readable storage medium of clause 11, the operations further comprising: computing a somatic coherence score representing similarity across non-verbal modalities, wherein computing the cross-modal affective divergence score is further based on the somatic coherence score.

Clause 19. The non-transitory computer-readable storage medium of clause 18, wherein computing the somatic coherence score comprises: computing a cosine similarity between each pair of non-verbal modalities; and computing an average of the cosine similarities between the pairs of non-verbal modalities as the somatic coherence score.

Clause 20. The non-transitory computer-readable storage medium of clause 11, the operations further comprising: applying a divergence characterization model to the pairwise divergence scores and the cross-modal affective divergence score to output a classification of divergence pattern exhibited in the multimodal data, wherein generating the personalized treatment plan is further based on the classification of the divergence pattern.

Clause 21. A computer-implemented method for causally-informed reasoning over longitudinal patient treatment data, comprising: maintaining a graph-based patient treatment record comprising nodes representing clinical events and edges representing relationships among the clinical events, wherein a subset of nodes represent treatment events and a subset of nodes represent outcome events; computing, by a propensity estimation module, for each treatment node in the graph, a propensity score representing an estimated probability that the treatment was selected given the patient’s clinical context prior to treatment selection, the propensity score being computed from a learned function of feature representations of nodes temporally preceding the treatment node; computing, by a graph attention network, attention weights between pairs of nodes; for edges connecting treatment nodes to subsequent outcome nodes, adjusting the attention weights by inverse propensity weights derived from the propensity scores to produce causally-adjusted attention weights that down-weight treatment-outcome correlations attributable to treatment selection confounding; performing message-passing over the graph using the causally-adjusted attention weights to produce causally-informed node embeddings; and generating a patient-level causal embedding from the causally-informed node embeddings, wherein the patient-level causal embedding encodes estimated causal relationships between treatments and outcomes.

Clause 22. The computer-implemented method of clause 21, wherein computing the causally-adjusted attention weight for an edge from treatment node j to outcome node k comprises: computing a product of a standard attention weight and an inverse propensity weight, wherein the inverse propensity weight is computed as one divided by a maximum of the propensity score and a clipping threshold; and renormalizing the causally-adjusted attention weights.

Clause 23. The computer-implemented method of clause 21, wherein the causal attention adjustment is selectively applied to edges connecting treatment nodes or medication nodes to assessment nodes, biometric nodes, or milestone nodes, and wherein standard attention weights are used for other edge types.

Clause 24. The computer-implemented method of clause 21, wherein the propensity estimation module is pre-trained on a treatment prediction task for a plurality of initial epochs before end-to-end training of a full architecture.

Clause 25. A computer-implemented method for federated learning across a plurality of distributed clinical nodes with heterogeneous psychiatric data distributions, comprising: computing, at each participating clinical node, a site-type embedding from statistics of the node’s local model parameter updates, the site-type embedding characterizing the node’s psychiatric data distribution without revealing individual patient data; clustering, at a central aggregation server, the site-type embeddings to identify groups of clinical nodes with similar psychiatric data distributions; computing, for each clinical node, a type-aware aggregation weight that incorporates both the node’s dataset size and a type-balancing factor ensuring that each identified group of sites receives equal aggregate influence on a global model; receiving, from each clinical node, a local model parameter update to which calibrated noise has been added to provide differential privacy guarantees; and computing an updated global model by aggregating the received local model parameter updates using the type-aware aggregation weights.

Clause 26. The computer-implemented method of clause 25, wherein the site-type embedding for a clinical node is computed from a mean and standard deviation of a local gradient update vector.

Clause 27. The computer-implemented method of clause 25, further comprising: computing per-layer aggregation weights, wherein for upstream model layers a standard data-proportional weight is used and for downstream model layers a stronger type-aware weight is used.

Clause 28. A computer-implemented method for safety-constrained pharmacological optimization using reinforcement learning, comprising: encoding, by a state encoder neural network, a pharmacological state comprising a patient’s pharmacogenomic profile, current medication regimen, and symptom severity into a state embedding; computing, by a neural network, raw action logits over a pharmacological action space; generating, by a differentiable constraint mask generator, a binary mask over the action space by evaluating a set of symbolic safety rules against the current pharmacological state, wherein the binary mask assigns zero to actions that violate any safety rule; applying the binary mask to the raw action logits before a softmax computation, such that actions violating safety rules receive zero probability in a policy output and gradient signal flows only through feasible actions; and training the state encoder and unconstrained action head using proximal policy optimization with a multi-objective reward function, wherein the constraint mask structurally excludes unsafe actions from policy gradient computation.

Clause 29. The computer-implemented method of clause 28, wherein the pharmacological state further comprises a causal patient embedding produced by a causally-informed graph attention network operating over a longitudinal patient treatment graph.

Clause 30. A system for detecting cross-modal affective divergence in a psychiatric assessment context, the system comprising: one or more processors; and a non-transitory computer-readable storage medium storing instructions that, when executed, cause the one or more processors to perform operations comprising: accessing multimodal biometric data from a patient captured by one or more sensors; encoding, by a plurality of modality-specific encoder neural networks, each modality of the multimodal biometric data into a modality-specific affective state representation; projecting, by learned modality-specific alignment projections trained with a contrastive alignment loss, each modality-specific affective state representation into a shared affective latent space; computing a pairwise divergence score between each pair of aligned modality representations in the shared affective latent space; computing a cross-modal affective divergence score representing the clinical significance of detected divergence based on the pairwise divergence scores; computing, based at least in part on the cross-modal affective divergence score, a per-modality trust weight indicating estimated reliability of each modality as an indicator of the patient’s affective state; generating a trust-weighted fused affective state estimate by combining the aligned modality representations according to the per-modality trust weights; and generating a personalized treatment plan based on the trust-weighted fused affective state.

### BRIEF DESCRIPTION OF THE DRAWINGS

FIG. 1 shows a networking environment for a mental healthcare system, according to some embodiments.

FIG. 2 shows a subsystem integration layer block diagram, according to some embodiments.

FIG. 3 shows a multimodal data processing flow diagram, according to some embodiments.

FIG. 4 shows a causal inference model architecture, according to some embodiments.

FIG. 5 shows a federated learning protocol diagram, according to some embodiments.

FIG. 6 shows an adaptive pharmacology delivery flowchart, according to some embodiments.

FIG. 7 shows a neural network architecture diagram, according to some embodiments.

FIG. 8 shows a transformer model architecture diagram, according to some embodiments.

The figures and flowcharts presented herein are illustrative of representative embodiments and are not intended to limit the scope of the invention to the specific configurations, arrangements, or sequences depicted. A person having ordinary skill in the art, upon review of the present disclosure, would readily understand the underlying principles governing the illustrated architectures and would be capable of implementing variations, modifications, and alternative embodiments that achieve the same functional results without departing from the scope of the invention as defined by the appended claims.

### DETAILED DESCRIPTION

### Overview

The present disclosure provides systems, methods, and non-transitory computer-readable media for closed-loop psychiatric clinical decision support using probabilistic state estimation and safety-constrained computation.

As a technological improvement to the traditional psychiatric assessment paradigm, the system maintains up-to-date state estimations for informing trajectory of the patient over time. This prevents session-to-session infrequency from creating any blind spots in the treatment of the patient.

### Networking Environment

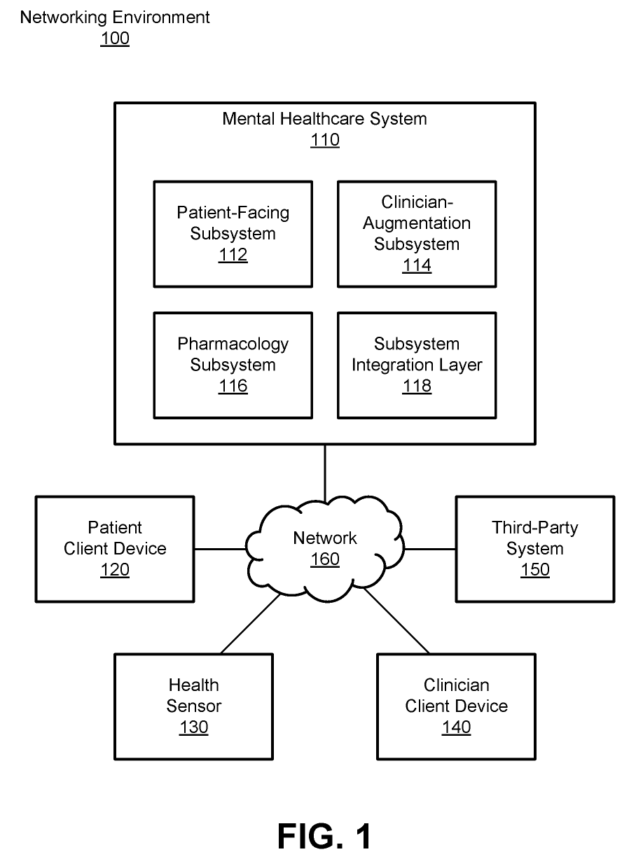

FIG. 1 shows a high-level system architecture diagram, according to some embodiments. The networking environment 100 comprises a mental healthcare system 110 that integrates multiple subsystems to provide comprehensive mental health services. The architecture supports real-time data synchronization and processing across distributed nodes while maintaining data privacy and security. The mental healthcare system 110 provides a unified platform for diagnostic assessment, therapeutic intervention, pharmacological optimization, clinical decision support, or some combinaion thereof.

The mental healthcare system 110 connects to external devices and systems through a network 160, enabling data exchange and communication across the ecosystem. The system 110 interfaces with a patient client device 120, one or more health sensors 130, a clinician client device 140, and a third-party system 150 to collect and process multimodal patient data.

The patient-facing subsystem 112 provides an interaction interface for patients to engage with the mental healthcare system 110. The patient-facing subsystem 112 may deliver AI-assisted therapeutic interventions, mood tracking, personalized mental health content through conversational interfaces and digital therapeutic tools, or some combination thereof.

The patient-facing subsystem 112 may be configured to collect multimodal biometric data from patients during therapeutic sessions, including text, voice, facial imagery, physiological signals, or some combination thereof. The patient-facing subsystem 112 can adapt therapeutic content in real time based on sentiment analysis, biometric feedback, longitudinal patient data, or some combination thereof. The patient-facing subsystem 112 integrates with the subsystem integration layer 118 to synchronize patient data with other components of the mental healthcare system 110.

The clinician-augmentation subsystem 114 functions as a real-time co-pilot for a mental healthcare provider during one or more clinical sessions. The clinician-augmentation subsystem 114provides automated session documentation, including SOAP notes, treatment plan updates, and diagnostic impressions generated from live session analysis. The clinician-augmentation subsystem 114 delivers evidence-based therapeutic recommendations informed by the patient’s longitudinal treatment history and current session context.

The clinician-augmentation subsystem 114 performs continuous risk assessment and generates alerts when crisis indicators are detected during clinical interactions. The clinician-augmentation subsystem 114 presents information through an interface that supports clinical decision-making without disrupting the therapeutic relationship.

The pharmacology subsystem 116 integrates pharmacogenomic data, gut-brain axis biomarkers, and longitudinal treatment response data to optimize psychiatric medication regimens. The pharmacology subsystem 116 generates personalized medication recommendations based on individual patient metabolizer phenotypes, drug-drug interactions, predicted treatment responses, or some combination thereof. The pharmacology subsystem 116 employs reinforcement learning algorithms to dynamically adjust dosing recommendations while maintaining safety constraints derived from clinical guidelines. The pharmacology subsystem 116 may further integrate with electronic health record systems through standardized APIs to access patient medication histories and laboratory results. The pharmacology subsystem 116 may generate explainable recommendations that trace the reasoning from genetic factors, biomarkers, or treatment history through to the final medication suggestion.

The pharmacology subsystem 116 generates personalized treatment plans that integrate pharmacogenomic analysis, gut-brain axis biomarkers, neural variability modeling, and longitudinal treatment response data. The pharmacology subsystem 116 employs safety-constrained reinforcement learning to optimize medication selection and dosing while ensuring all recommendations comply with symbolic safety rules derived from clinical guidelines, drug interaction databases, and pharmacogenomic contraindications. The pharmacology subsystem 116 can continuously monitor treatment outcomes and updates its recommendations based on observed patient responses, adherence patterns, side effect profiles, or some combination thereof.

The pharmacology subsystem 116 presents recommended treatment plans to a clinician through a decision support interface. The recommended treatment is presented with explainability metadata, contributing genetic factors, biomarker considerations, predicted treatment responses, alternative treatment options, or some combination thereof. The pharmacology subsystem 116 requires explicit clinician approval of each treatment plan before execution, maintaining human oversight of all pharmacological interventions.

The decision support interface displays the treatment plan with comprehensive supporting information organized into multiple presentation layers. The interface presents a primary recommendation panel showing the suggested medication, dosage, administration schedule, and expected timeline to therapeutic effect. The interface includes an evidence summary panel that synthesizes the pharmacogenomic analysis results, displaying the patient’s metabolizer phenotype for relevant cytochrome P450 enzymes and explaining how these genetic factors influence the medication recommendation. The interface presents gut-brain axis considerations including microbiome composition indicators and their predicted impact on drug metabolism and neurotransmitter synthesis. The interface displays neural variability model outputs showing the estimated receptor density profiles, circuit connectivity parameters, and neuroplasticity factors that informed the recommendation.

The interface provides a comparative efficacy panel presenting predicted outcome distributions for the recommended treatment and alternative treatment options, displayed as probability distributions over symptom improvement scores at multiple time horizons. The interface includes a side effect risk assessment showing predicted probabilities for common and serious adverse effects based on the patient’s pharmacogenomic profile and treatment history. The interface presents drug interaction analysis results identifying any potential interactions with the patient’s current medications, supplements, or medical conditions, with each interaction categorized by severity level and clinical significance. The interface displays adherence prediction metrics estimating the likelihood of treatment adherence based on factors including dosing complexity, side effect burden, patient preference data, and historical adherence patterns.

The interface includes an explainability panel that traces the reasoning pathway from input data through intermediate computational steps to the final recommendation, showing the relative contribution of pharmacogenomic factors, gut-brain axis modeling, neural variability optimization, and reinforcement learning policy outputs. The interface presents safety constraint information identifying which symbolic safety rules were evaluated, which constraints were active for this patient, and which alternative treatment options were excluded due to contraindications or safety concerns. The interface provides access to supporting clinical evidence including relevant clinical trial data, practice guideline recommendations, and population-level treatment response statistics that support the recommendation. The interface enables the clinician to explore alternative treatment scenarios through interactive simulation, adjusting proposed medications or dosages and viewing updated outcome predictions and risk assessments in real time.

The subsystem integration layer 118 provides the computational infrastructure that enables interoperability among the patient-facing subsystem 112, clinician-augmentation subsystem 114, and pharmacology subsystem 116. The layer 118 comprises a unified data ontology that standardizes mental health entities across all subsystems using healthcare interoperability protocols. The subsystem integration layer 118 operates a real-time event bus that facilitates asynchronous communication and data synchronization among the three primary subsystems. The layer 118 includes a cross-system inference engine that evaluates events from multiple subsystems collectively to generate composite clinical insights. The subsystem integration layer 118 maintains federated learning capabilities, differential privacy protections, blockchain-secured audit trails, including other functionality for interoperability.

The patient client device 120 provides an interface through which patients interact with the patient-facing subsystem 112. The patient client device 120 may be a mobile device, a tablet, a laptop computer, a desktop computer, a web browser application executed on a computing device, or another computing device. The patient client device 120 captures multimodal input data including text entries, voice audio recordings, video imagery for facial expression analysis, or some combination thereof. The patient client device 120 transmits collected data to the mental healthcare system 110 through secure, encrypted communication channels via the network 160.

The patient client device 120 receives and displays a user interface generated by the patient-facing subsystem 112. The user interface may present content in conjunction with mental healthcare applications, e.g., providing therapeutic content, assessment instruments, personalized interventions, crisis prevention resources, or some combination thereof from the patient-facing subsystem 112.

The one or more health sensors 130 acquire physiological and behavioral data from patients for integration into the mental healthcare system 110. The health sensors 130 may comprise wearable devices that monitor heart rate variability, galvanic skin response, sleep architecture, physical activity levels, or some combination thereof. The health sensors 130 may integrate with the mental healthcare system 110 through standardized APIs including Apple HealthKit, Google Fit, Fitbit Web API, or some combination thereof. The health sensors 130 transmit biometric measurements to the mental healthcare system 110 for processing by sentiment analysis modules, crisis prediction algorithms, or some combination thereof. The health sensors 130 enable continuous monitoring of patient physiological states outside of scheduled clinical interactions.

The clinician client device 140 provides mental healthcare providers with access to the clinician-augmentation subsystem 114, e.g., before, during, or after clinical sessions. The clinician client device 140 may be a mobile device, a laptop computer, a desktop computer, a tablet, or another computing device. The clinician client device 140 provides an interface to give non-intrusive access during therapeutic interactions.

The clinician client device 140 can present real-time session analysis, therapeutic recommendations, risk assessment alerts, automated documentation, or some combination thereof from the clinician-augmentation subsystem 114. The clinician client device 140 enables clinicians to review, edit, or approve AI-generated recommendations, treatment plans, or clinical notes. The clinician client device 140 maintains secure authentication and access control to protect patient data in accordance with regulatory requirements.

The third-party system 150 comprises external healthcare systems, electronic health record platforms, laboratory information systems, pharmacogenomic testing services, or some combination thereof that exchange data with the mental healthcare system 110. The third-party system 150 may transmit patient demographic information, diagnostic codes, medication histories, laboratory results, pharmacogenomic test results, or some combination thereof to the mental healthcare system 110 through standardized healthcare interoperability protocols. The third-party system 150 receives clinical documentation, treatment recommendations, risk assessments, or some combination thereof from the mental healthcare system 110 for integration into external clinical workflows. The third-party system 150 communicates with the mental healthcare system 110 through secure data exchange protocols. The third-party system 150 enables the mental healthcare system 110 to function as an integrated component within the broader healthcare delivery ecosystem.

The network 160 provides communication infrastructure connecting the mental healthcare system 110 with the patient client device 120, the one or more health sensors 130, the clinician client device 140, the third-party system 150, or some combination thereof. The network 160 may comprise the Internet, local area networks, wide area networks, cellular networks, wireless networks, or some combination thereof. The network 160 employs encryption protocols including TLS, end-to-end encryption, or some combination thereof to secure data transmission and protect patient privacy. The network 160 supports real-time data streaming for live session analysis, asynchronous data transfer for federated learning model updates, event-driven communication through the real-time event bus, or some combination thereof. The network 160 maintains sufficient bandwidth and latency characteristics to enable responsive therapeutic interactions and timely crisis intervention capabilities.

### Subsystem Integration Layer

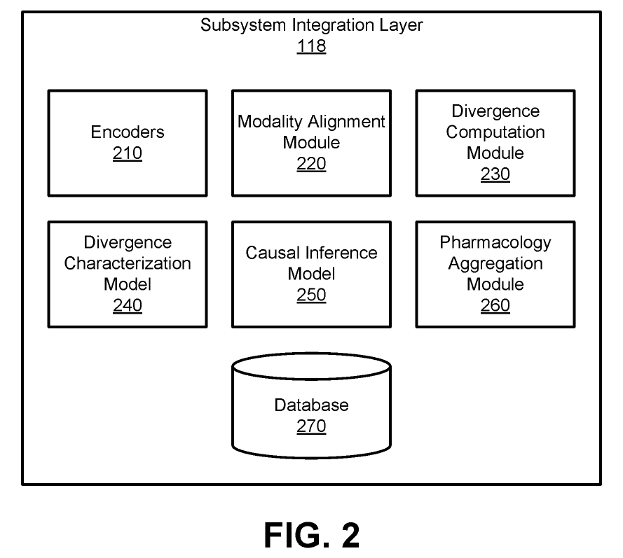

FIG. 2 shows a subsystem integration layer block diagram, according to some embodiments. The subsystem integration layer 118 provides interoperability between the disparate subsystems in the mental healthcare system 110. As embodied in FIG. 2, the subsystem integration layer 118 includes one or more encoders 210, a modality alignment module 220, a divergence computation module 230, a divergence characterization model 240, a causal inference model 250, a pharmacology aggregation module 250, a governance module 260, and a database 270. In other embodiments, the subsystem integration layer 118 may include additional, fewer, or different components than those listed herein. In further embodiments, the functionality can be disparately distributed throughout the modules or subsystems. For example, the governance module 260 can be deployed on each subsystem for validation of recommendations or computational analyses.

The one or more encoders 210 encode multimodal input data from disparate sources to generate embeddings for downstream analyses. The encoders 210 comprise a plurality of modality-specific encoder neural networks, each configured to process a distinct data modality and produce a corresponding embedding representation. The encoders 210 may include a text encoder that processes conversational text input through a transformer-based language model to produce a text-derived affective state vector. The encoders 210 may include a voice encoder that processes voice audio features including Mel-frequency cepstral coefficients, fundamental frequency, jitter, shimmer, and speaking rate extracted from audio sampled at a minimum of 16 kHz to produce a voice-derived affective state vector. The encoders 210 may include a face encoder that processes facial action unit activation vectors detected from video imagery at a minimum of 30 frames per second through a convolutional neural network to produce a face-derived affective state vector. The encoders 210 may include a physiology encoder that processes physiological time series data including heart rate variability from inter-beat intervals, galvanic skin response, and respiratory rate through a temporal convolutional network to produce a physiology-derived affective state vector. Each encoder 210 is trained to map its respective modality-specific input into a representation of the patient’s affective state as expressed through that modality, with each encoder producing an embedding of dimensionality d. The encoders 210 are trained independently before alignment such that each encoder learns the affective information content specific to its modality without being influenced by other modalities during encoding.

The modality alignment module 220 aligns the embeddings generated by the disparate encoders such that the embeddings are mapped into a shared latent space. The modality alignment module 220 projects each modality-specific affective state representation into a shared affective latent space using learned alignment projections. The modality alignment module 220 comprises modality-specific learned linear projections that transform each encoder output into the shared space. The modality alignment module 220 trains the alignment projections using a contrastive alignment loss that encourages aligned representations to cluster together when modalities convey concordant affective states and to separate when modalities convey discordant affective states. The contrastive alignment loss computes similarity between aligned representations from different modalities for the same patient and contrasts these with representations from other patients in the training batch.

The modality alignment module 220 preserves inter-modal disagreement rather than resolving it, such that downstream modules can detect clinically significant divergence between the various patient cues. The modality alignment module 220 produces aligned embeddings for each modality that maintain the same dimensionality as the encoder outputs while empowering direct comparison across modalities in the shared latent space.

The divergence computation module 230 computes explicit divergence features from the aligned modality representations produced by the modality alignment module 220.

The divergence computation module 230 computes, for each pair of modalities, a pairwise divergence score based in part on the cosine similarity between the aligned representations in the shared affective latent space. In particular, the divergence score can be computed as one minus the cosine similarity, or effectively the complement of the cosine similarity. By computing pairwise divergence, the divergence computation module 230 generates a divergence matrix where each entry quantifies the degree of disagreement between two modalities. The divergence computation module 230 calculates divergence scores for all possible modality pairs, including text-voice divergence, text-face divergence, text-physiology divergence, voice-face divergence, voice-physiology divergence, and face-physiology divergence. The divergence matrix provides a comprehensive characterization of inter-modal agreement and disagreement patterns across the multimodal input data.

The divergence computation module 230 computes these pairwise divergence scores using the formula div(m1, m2) = 1 - cosine_similarity(a_m1, a_m2), where a_m1 and a_m2 are the aligned representations for modalities m1 and m2 respectively. The divergence scores range from 0 to 2, where values near 0 indicate high concordance between modalities, values near 1 indicate orthogonal or unrelated affective signals, and values near 2 indicate opposing affective signals. The divergence computation module 230 generates the divergence matrix as a symmetric matrix with dimensionality M×M, where M is the number of active modalities, with each matrix element representing the divergence between a corresponding pair of modalities.

The divergence computation module 230 computes a verbal-non-verbal divergence score (VNDS) as the maximum divergence between the textual modality and any non-textual modality, capturing the degree to which a patient’s verbal expression diverges from their physiological, vocal, or facial signals.

The VNDS can be computed as VNDS = max(div(text, voice), div(text, face), div(text, physiology)), where each pairwise divergence is calculated as described above. For example, consider a patient who verbally reports "I’m doing fine" during a therapeutic session. The text encoder produces an aligned representation a_text indicating neutral-to-positive affect. Simultaneously, the voice encoder detects reduced pitch variability and increased vocal tension, producing an aligned representation a_voice potentially indicating distress. The face encoder detects facial action units, e.g., brow lowering and lip corner depression indicating sadness, producing an aligned representation a_face also indicating distress. The physiology encoder detects elevated galvanic skin response and reduced heart rate variability, producing an aligned representation a_physio indicating physiological arousal.

The divergence computation module 230 may compute example pairwise divergence scores div(text, voice) = 0.78, div(text, face) = 0.82, and div(text, physiology) = 0.71. The VNDS is computed as max(0.78, 0.82, 0.71) = 0.82, indicating substantial divergence between the patient’s verbal report and their non-verbal signals. This high VNDS value flags a clinically significant pattern wherein the patient’s explicit verbal expression does not align with their implicit physiological and behavioral indicators, suggesting potential affective masking or minimization that warrants clinical attention.

The divergence computation module 230 computes a somatic coherence score (SCS) as the average similarity among non-textual modalities, measuring the degree to which physiological, vocal, and facial signals are internally consistent with one another. The SCS can be calculated as SCS = mean(sim(a_voice, a_face), sim(a_voice, a_physio), sim(a_face, a_physio)), where sim() denotes cosine similarity between aligned representations. The SCS ranges from -1 to 1, where values near 1 indicate high internal coherence among non-verbal modalities, values near 0 indicate independence, and values near -1 indicate internal contradiction among non-verbal signals.

Continuing the numerical example from the VNDS computation above, the divergence computation module 230 computes the somatic coherence score for the same patient scenario. Given the aligned representations a_voice (indicating distress from vocal tension), a_face (indicating sadness from facial action units), and a_physio (indicating physiological arousal from elevated galvanic skin response), the module computes pairwise similarities: sim(a_voice, a_face) = 0.86, sim(a_voice, a_physio) = 0.91, and sim(a_face, a_physio) = 0.87. The SCS is computed as mean(0.86, 0.91, 0.87) = 0.88, indicating high internal coherence among the non-verbal modalities. This high SCS value of 0.88, combined with the previously computed high VNDS value of 0.82, provides strong computational evidence of affective masking: the patient’s verbal report diverges substantially from their non-verbal signals, while the non-verbal signals themselves are highly coherent with one another, all indicating distress. This pattern suggests that the patient is consciously or unconsciously minimizing their distress in verbal communication while their physiological, vocal, and facial channels consistently express the underlying affective state.

A high verbal-non-verbal divergence score combined with a high somatic coherence score indicates a clinically significant pattern wherein the patient’s verbal expression diverges from their non-verbal signals while the non-verbal signals are internally coherent, providing a computational signature of affective masking or minimization.

The divergence computation module 230 provides these divergence features as input to the divergence characterization model 240 for further clinical interpretation.

The divergence characterization model 240 is a computational model that inputs the divergence features (e.g., the divergence matrix, the VNDS, the SCS, or some combination thereof) to classify the patient’s psychological state. In one or more embodiments, the divergence characterization model 240 is implemented as a rule-based decision tree that parses branched decision nodes to classify the divergence pattern. In such embodiments, each divergence characterization model 240 may evaluate the inputs against the logical operands in the decision tree to determine which path to traverse along the decision tree.

The divergence characterization model 240 classifies psychological states based on the detected divergence patterns. For example, the model may classify a state as "masking" when a patient exhibits high verbal-non-verbal divergence with high somatic coherence, indicating conscious suppression of distress signals in verbal communication while non-verbal channels consistently express the underlying negative affect. The model may classify a state as "minimization" when a patient verbally downplays symptom severity while physiological and behavioral indicators suggest more significant distress than acknowledged. The model may classify a state as "alexithymia" when divergence patterns suggest difficulty identifying or describing emotional states, characterized by low correlation between verbal emotional labels and corresponding physiological arousal patterns. The model may classify a state as "dissociation" when the patient’s verbal content appears disconnected from affective expression across all non-verbal modalities, with flat or incongruent physiological responses to emotionally charged verbal content. The model may classify a state as "incongruent positive affect" when facial expressions or vocal prosody suggest positive affect that contradicts verbal reports of distress or physiological indicators of negative arousal. The model may classify a state as "no significant divergence" when all modalities demonstrate concordant affective signals with low pairwise divergence scores across all modality pairs.

In some embodiments, the divergence characterization model 240 may be a supervised machine learning model trained on clinician-annotated data to learn the mapping from divergence features to clinically significant divergence patterns. The divergence characterization model 240 may be implemented with a feedforward neural network that takes as input the concatenation of the divergence matrix (flattened), the VNDS, the SCS, the individual aligned representations from each modality, temporal context features representing the rate of change of divergence scores over a preceding time window, or some combination thereof. The divergence characterization model 240 outputs a characterization or classification of the patient’s psychological state based on the input data.

The divergence characterization model 240 is trained using supervised learning on a dataset of multimodal psychiatric assessment sessions where trained clinicians have annotated each session for the presence and type of cross-modal affective divergence. During training, the model learns to associate specific patterns in the divergence features with clinician-identified divergence types including masking, minimization, alexithymia, dissociation, incongruent positive affect, or no significant divergence. The training employs a multi-task loss function that combines binary cross-entropy for divergence detection, cross-entropy for divergence type classification, mean squared error for trust-weighted fusion accuracy, and a regularization term to maintain alignment quality. The supervised training process enables the divergence characterization model 240 to generalize from the clinician-annotated training examples to accurately detect and classify divergence patterns in new patient data.

The causal inference model 250 performs causally-informed reasoning over longitudinal patient treatment data to distinguish causal relationships from correlational patterns. The causal inference model 250 operates on a patient graph that can represent the patient’s treatment history as a graph structure with nodes representing clinical events and edges representing relationships among those events. The causal inference model 250 addresses the technical problem that standard temporal graph neural networks learn correlations between treatments and outcomes without accounting for treatment selection bias, wherein patients who receive particular treatments differ systematically from patients who do not receive those treatments due to clinical features that also affect outcomes.

According to some implementations, the causal inference model 250 includes an attention network component that computes attention weights between nodes in the patients graph, a propensity network that estimates the probability that each treatment was selected given the patient’s clinical context prior to treatment selection, or some combination thereof. The propensity network processes feature representations of all nodes temporally preceding each treatment node to generate a propensity score representing the estimated likelihood of treatment selection. The causal inference model 250 adjusts the attention weights computed by the attention network using inverse propensity weighting, such that treatment-outcome correlations attributable to treatment selection confounding are down-weighted while genuine causal effects are preserved or up-weighted.

The causal inference model 250 applies causal attention adjustment selectively to edges connecting treatment nodes to subsequent outcome nodes, while maintaining standard attention computation for other edge types including temporal edges between assessment nodes and edges between biometric measurements. The causal inference model 250 performs message-passing over the graph using the causally-adjusted attention weights to produce causally-informed node embeddings that encode estimated causal relationships rather than confounded correlations. The causal inference model 250 generates a patient-level causal embedding from the causally-informed node embeddings through a graph-level readout function, providing downstream components with treatment history information that has been deconfounded for treatment selection bias.

The causal inference model 250 is trained using a multi-task loss function that combines a primary outcome prediction task with a propensity estimation task and a regularization term that penalizes extreme causal attention weights to maintain training stability. The propensity network component of the causal inference model 250 is pre-trained on a treatment prediction task before end-to-end training of the full architecture, ensuring that propensity estimates are reasonably calibrated before they are used for causal adjustment. The causal inference model 250 provides causally-informed patient embeddings to the pharmacology aggregation module 250 for use in treatment optimization, to the digital twin system for simulation of intervention outcomes, or to other components requiring deconfounded treatment effect estimates.

The pharmacology aggregation module 250 integrates pharmacogenomic data, gut-brain axis biomarkers, neural variability measurements, ]longitudinal treatment response datam or some combination thereof to generate personalized psychopharmacological recommendations. The pharmacology aggregation module 250 receives pharmacogenomic test results including cytochrome P450 enzyme polymorphism profiles that determine metabolizer phenotypes for medications metabolized through CYP2D6, CYP2C19, CYP3A4, CYP1A2, CYP2B6, or some combination thereof. The pharmacology aggregation module 250 processes genetic markers including serotonin transporter gene variants, brain-derived neurotrophic factor polymorphisms, dopamine receptor variants, COMT variants, HLA alleles relevant to drug hypersensitivity, or some combination thereof. The pharmacology aggregation module 250 applies a Bayesian network model that integrates these genetic markers with patient demographics and clinical history to generate probabilistic medication response profiles for candidate medications.

The pharmacology aggregation module 250 incorporates gut-brain axis data including microbiome composition data obtained from 16S rRNA sequencing or metagenomic analysis, gastrointestinal biomarkers including intestinal permeability markers and short-chain fatty acid levels, nutritional status indicators, or some combination thereof. The pharmacology aggregation module 250 models the influence of gut microbiota on neurotransmitter precursor availability, drug absorption and metabolism through cytochrome expression modulated by microbial metabolites, neuroinflammatory pathways mediated by gut-derived compounds, or some combination thereof. These gut-brain axis influences are encoded as probabilistic modifiers applied to the medication response profiles generated from pharmacogenomic analysis.

The pharmacology aggregation module 250 employs neural variability modeling to capture individual differences in neural circuit dynamics, neuroplasticity, and neurotransmitter receptor sensitivity. The pharmacology aggregation module 250 constructs patient-specific neural state models comprising estimated neurotransmitter receptor density profiles derived from pharmacogenomic data and treatment response history, neural circuit connectivity estimates derived from behavioral and cognitive assessment data, neuroplasticity parameters estimated from treatment response trajectories, or some combination thereof. The pharmacology aggregation module 250 uses optimization methods operating in high-dimensional parameter spaces to identify optimal medication-dose combinations that maximize predicted therapeutic benefit.

The pharmacology aggregation module 250 continuously adjusts medication dosing recommendations using a reinforcement learning framework. The reinforcement learning agent operates with a state representation comprising the pharmacogenomic profile, current medication regimen, symptom severity vector, side effect vector, biometric indicators, adherence rate, time on current regimen, causal patient embeddings from the causal inference model 250, or some combination thereof. The reinforcement learning agent proposes dosing adjustments including dose increases, dose decreases, dose maintenance, medication class switches, addition of augmentation agents, removal of augmentation agents, or some combination thereof. The reinforcement learning agent employs a multi-objective reward function that balances symptom improvement, side effect burden minimization, adherence probability maximization, drug interaction risk minimization, or some combination thereof with clinically configured priority weights.

The pharmacology aggregation module 250 validates all reinforcement learning-recommended actions against symbolic safety rules derived from the governance module 260 before presenting recommendations to clinicians. The pharmacology aggregation module 250 generates recommendations with explainability metadata that traces the reasoning from contributing genetic factors, gut-brain axis considerations, neural variability model predictions, reinforcement learning policy rationale, or some combination thereof through to the final medication suggestion. The pharmacology aggregation module 250 presents all outputs to treating clinicians as decision support recommendations requiring explicit clinician approval before execution, maintaining human oversight of all pharmacological interventions.

The governance module 260 enforces safety constraints and regulatory compliance across all AI-generated recommendations within the mental healthcare system 110. The governance module 260 comprises a clinical knowledge graph encoding symbolic safety rules derived from FDA drug labeling, pharmacological interaction databases, evidence-based clinical practice guidelines, regulatory requirements, or some combination thereof.

The governance module 260 validates all treatment recommendations generated by the pharmacology aggregation module 250 against contraindication rules, maximum dose limits, drug-drug interaction rules, drug-gene interaction rules based on metabolizer phenotypes, minimum therapeutic trial duration requirements, or some combination thereof before recommendations are presented to clinicians.

The governance module 260 implements a rule-based constraint checking system that evaluates proposed pharmacological actions against the encoded safety rules and rejects any actions that violate hard constraints. The governance module 260 provides explainability metadata for all constraint violations, identifying the specific safety rules that were triggered and the clinical rationale for each constraint.

The governance module 260 maintains audit logs of all constraint checks, approved recommendations, rejected recommendations, or some combination thereof on the blockchain-secured distributed ledger to ensure regulatory compliance and enable retrospective review. The governance module 260 supports configurable rule sets that can be customized per clinical site, patient population, regulatory jurisdiction, or some combination thereof while maintaining a core set of universal safety constraints.

The database 270 stores patient data, clinical records, AI model parameters, training datasets, audit logs, or some combination thereof. The database 270 maintains longitudinal patient treatment histories including diagnostic assessments, therapeutic interventions, medication events, biometric measurements, crisis events, treatment milestones, or some combination thereof. The database 270 stores multimodal biometric data including text transcripts, voice audio features, facial imagery, physiological sensor readings, behavioral metadata, or some combination thereof collected during patient interactions with the mental healthcare system 110. The database 270 maintains pharmacogenomic profiles including cytochrome P450 enzyme polymorphism data, serotonin transporter gene variants, brain-derived neurotrophic factor polymorphisms, dopamine receptor variants, HLA alleles, or some combination thereof for personalized medication optimization. The database 270 stores gut-brain axis biomarkers including microbiome composition data, gastrointestinal markers, nutritional status indicators, or some combination thereof. The database 270 maintains federated learning model parameters, gradient updates, privacy budget tracking data, or some combination thereof to support decentralized AI model training. The database 270 stores clinical knowledge graph data including diagnostic criteria, treatment protocols, medication interaction rules, contraindication rules, escalation rules, or some combination thereof. The database 270 maintains blockchain ledger references, smart contract states, patient consent records, or some combination thereof for audit trail and compliance purposes. The database 270 implements encryption at rest, access control policies, data retention policies, or some combination thereof to ensure data security and regulatory compliance with HIPAA, GDPR, or other applicable regulations.

### Multimodal Data Processing For Divergence Characterization

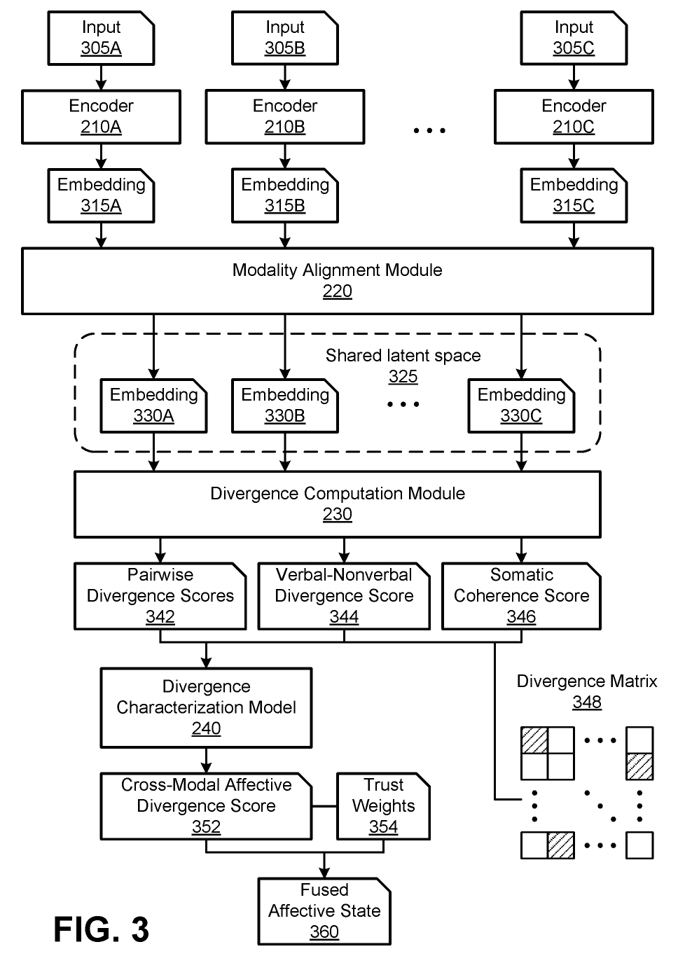

FIG. 3 shows a multimodal data processing flow diagram, according to some embodiments. FIG. 3 is an illustrative workflow of the modules of the subsystem integration layer 118 in characterizing divergence patterns from multimodal input data.

The pipeline begins with multiple input streams 305A, 305B, 305C representing distinct data modalities, e.g., collected from a patient during a session, a therapeutic event, or another recorded opportunity. Each input stream is processed by a corresponding modality-specific encoder 210A, 210B, 210C that transforms the raw input data into a learned representation. Each encoder 210A, 210B, 210C generates a modality-specific embedding 315A, 315B, 315C that represents the patient’s affective state as expressed through that particular modality.

The modality-specific embeddings 315A, 315B, 315C are input to the modality alignment module 220, which projects each embedding into a shared latent space 325 using learned alignment projections. The modality alignment module 220 applies contrastive learning to train the alignment projections such that embeddings from concordant modalities cluster together in the shared space while embeddings from discordant modalities separate. The aligned embeddings 330A, 330B, 330C in the shared latent space 325 preserve inter-modal disagreement rather than resolving it, enabling downstream detection of clinically significant divergence patterns.

The divergence computation module 230 receives the aligned embeddings 330A, 330B, 330C and computes quantitative divergence metrics including pairwise divergence scores 342 between each pair of modalities, a verbal-nonverbal divergence score 344 measuring the maximum divergence between textual and non-textual modalities, a somatic coherence score 346 measuring the internal consistency among non-textual modalities, or some combination thereof. The divergence computation module 230 generates a divergence matrix 348 that comprehensively characterizes the agreement and disagreement patterns across all modality pairs.

The divergence characterization model 240 receives the divergence metrics from the divergence computation module 230 including the pairwise divergence scores 342, the verbal-nonverbal divergence score 344, the somatic coherence score 346, the divergence matrix 348, or some combination thereof.

The divergence characterization model 240 processes these divergence features along with the aligned embeddings 330A, 330B, 330C and temporal context information to generate a cross-modal affective divergence score 352 representing the clinical significance of the detected divergence. The divergence characterization model 240 computes per-modality trust weights 354 indicating the estimated reliability of each modality as an indicator of the patient’s true affective state given the detected divergence pattern. The trust weights 354 are applied to the aligned embeddings 330A, 330B, 330C to produce a fused affective state 360 that represents the patient’s estimated true affective state by weighting more reliable modalities more heavily and down-weighting modalities that exhibit divergence from the underlying affective state.

### Casual Inference In Patient Graph

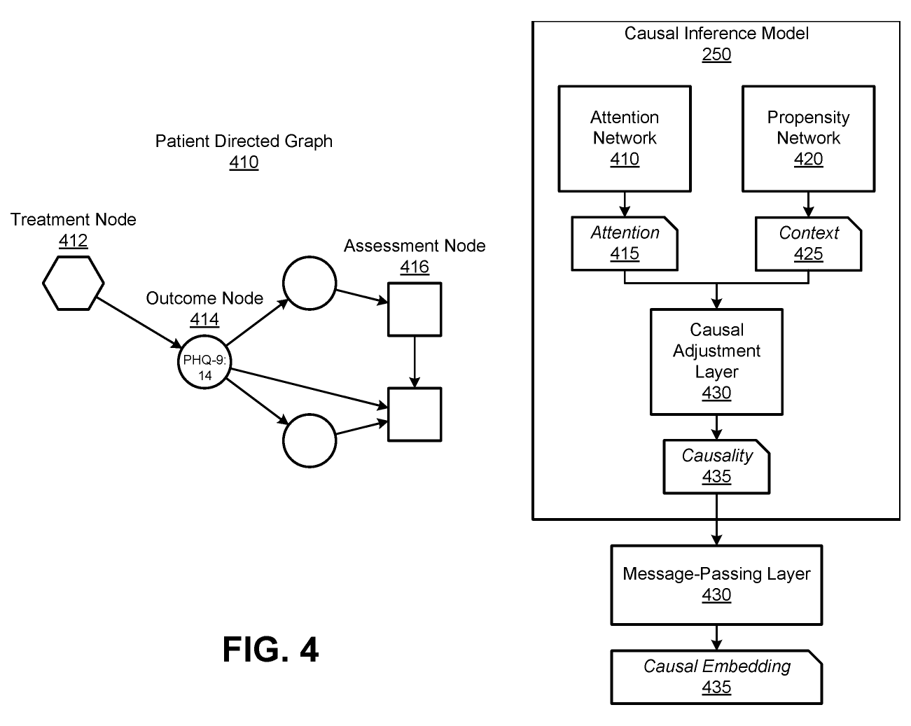

FIG. 4 shows a causal inference model architecture, according to some embodiments. FIG. 4 is an illustrative workflow of the causal inference model 250 of the subsystem integration layer 118 in determining causal relations.

The patient directed graph 410 comprises nodes representing clinical events including treatment nodes 412, outcome nodes 414, and assessment nodes 416, connected by directed edges representing temporal and clinical relationships.

The causal inference model 250 comprises an attention network 410 that computes standard attention weights between nodes based on their feature representations and temporal relationships, and a propensity network 420 that estimates the probability of treatment selection given the patient’s clinical context. The attention network 410 processes node features through learned transformations to generate attention scores that quantify the relevance of each node to downstream nodes in the graph. The propensity network 420 analyzes features from nodes temporally preceding each treatment node to generate propensity scores representing the estimated likelihood that each treatment was selected given the patient’s clinical state at the time of treatment.

The causal inference model 250 further comprises a causal adjustment layer 430 that modifies the attention weights computed by the attention network 410 using inverse propensity weighting derived from the propensity scores generated by the propensity network 420. The causal adjustment layer 430 applies the adjustment selectively to edges connecting treatment nodes to subsequent outcome nodes, down-weighting treatment-outcome correlations that are likely attributable to treatment selection confounding while preserving or up-weighting correlations that represent genuine causal effects.

The causal adjustment layer 430 combines these inputs to produce causally-adjusted attention weights that are used in message-passing operations to generate causally-informed node embeddings. The causal inference model 250 outputs causality 435 information in the form of deconfounded embeddings that encode estimated causal relationships rather than confounded correlations, providing downstream components with treatment history information that accounts for treatment selection bias.

### Federated Learning Protocol

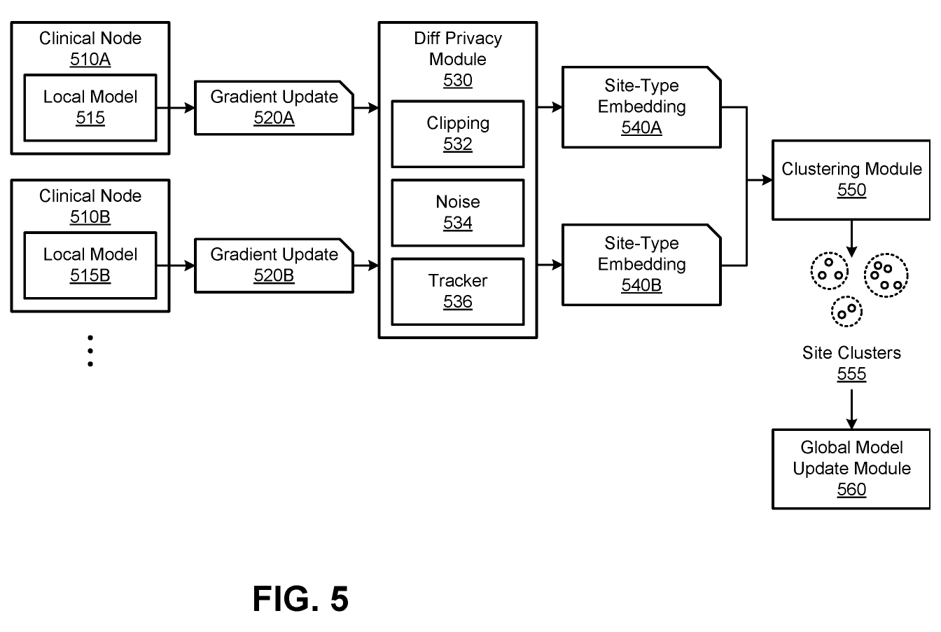

FIG. 5 shows a federated learning protocol employed by the subsystem integration layer 118, according to some embodiments. The architecture comprises multiple clinical nodes, e.g., clinical nodes 510A, 510B, and so on, each maintaining a local model 515 and 515B that is trained on locally stored patient data. Each clinical node performs local training for a configured number of epochs using gradient-based optimization, generating local model parameter updates that reflect the learning from that site’s patient population. The local model updates are processed through a differential privacy module 530 before transmission to ensure mathematical privacy guarantees.

The differential privacy module 530 applies privacy-preserving transformations to the local model updates before they are transmitted to the central aggregation infrastructure. The module 530 comprises a clipping component 532 that bounds the L2 norm of each gradient update to a maximum threshold, preventing any single patient’s data from having disproportionate influence on the model update. The module 530 further comprises a noise component 534 that adds calibrated Gaussian noise to the clipped gradients, with the noise magnitude determined by the configured privacy parameters epsilon and delta. The module 530 includes a tracker component 536 that monitors cumulative privacy loss across training rounds using privacy accounting methods, ensuring that the total privacy budget is not exceeded over the course of federated training. The privatized model updates are transmitted from each clinical node through encrypted communication channels to the aggregation infrastructure.

The transmitted updates from clinical nodes 510A and 510B are processed by site-type embedding modules 540A and 540B that characterize each site’s data distribution without accessing raw patient data. The site-type embeddings are computed from statistics of the gradient updates including mean, standard deviation, entropy of prediction distributions, skewness, or some combination thereof, capturing the statistical character of each site’s patient population. The site-type embeddings from multiple clinical nodes are input to a clustering module 550 that identifies groups of sites with similar psychiatric data distributions, generating site clusters 555 that represent distinct site-types such as anxiety-focused clinics, depression-focused clinics, inpatient facilities, or other clinical specializations. The identified site clusters 555 are used by a global model update module 560 to compute type-aware aggregation weights that balance the influence of different site-types on the global model, ensuring that the federated model performs well across all clinical contexts rather than being dominated by majority site-types. The global model update module 560 combines the privatized updates from all participating sites using the type-aware weights to produce an updated global model that is distributed back to the clinical nodes for the next training round.

### Example Method

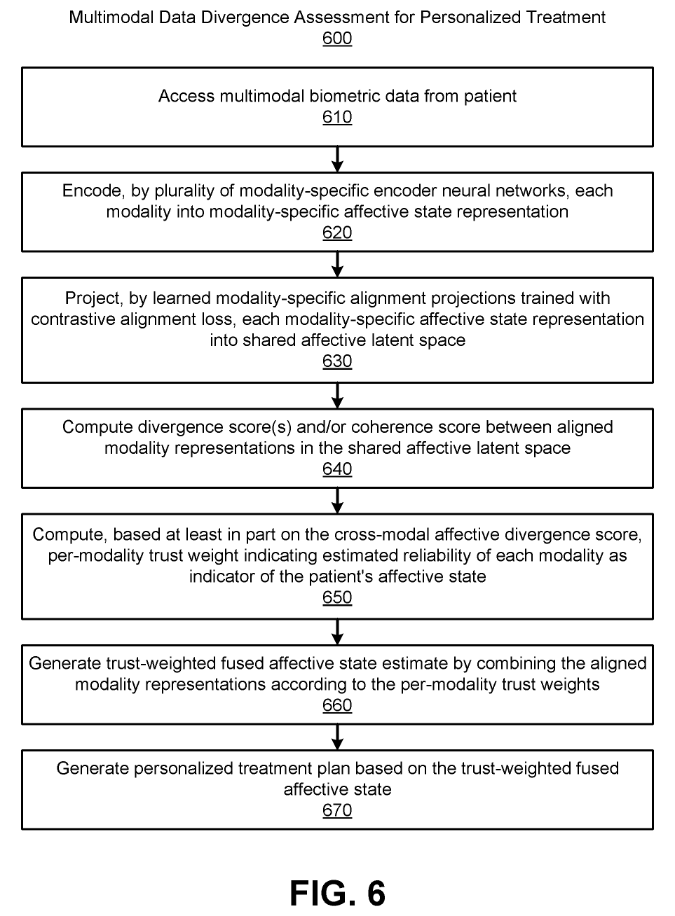

FIG. 6 shows an adaptive pharmacology delivery flowchart, according to some embodiments. FIG. 6 is described as performed by a system (e.g., the mental healthcare system 110). In some implemenations, the subsystem integration layer 118 may perform the various steps, in conjunction with other subsystems of the mental healthcare system 110. Other embodiments may include additional, fewer, or different steps than those listed herein.

The system accesses 610 multimodal biometric data from a patient. This may include text, voice, facial imagery, physiological signals, or some combination thereof collected by health sensors during therapeutic interactions.

The system encodes 620, via a plurality of encoders, each modality into modality-specific affective state representations. The encoders can be independently-trained neural networks trained to capture affective information content specific to each modality. Each encoder processes its respective input stream to generate embeddings representing the patient’s affective state as expressed through that particular modality. The encoders 210 of the subsystem integration layer 118 may perform the modality-specific encoding.

The system projects 630 each modality-specific representation into a shared affective latent space using learned alignment projections trained with contrastive alignment loss. The alignment preserves inter-modal disagreement rather than resolving it, enabling detection of clinically significant divergence patterns between concordant and discordant affective signals. The modality alignment module 220 may perform the projection into the shared latent space.

The system computes 640 one or more divergence scores or a coherence score between aligned modality representations in the shared latent space. The module generates pairwise divergence scores, verbal-nonverbal divergence scores, somatic coherence scores, or some combination thereof that quantify agreement and disagreement patterns across modalities. The divergence computation module 230 may compute the divergence scores or the coherence score.

The system computes 650 per-modality trust weights based on the cross-modal affective divergence score. The trust weights indicate estimated reliability of each modality as an indicator of the patient’s true affective state, accounting for detected divergence patterns including masking or minimization. The divergence characterization module 240 may compute the per-modality trust weights.

The system generates 660 a trust-weighted fused affective state estimate by combining aligned modality representations according to computed trust weights. The fusion weights reliable modalities more heavily while down-weighting modalities exhibiting divergence, producing an accurate representation of the patient’s underlying affective state.

The system generates 670 a personalized treatment plan based on the trust-weighted fused affective state. The treatment plan integrates the divergence-informed affective assessment with pharmacogenomic data, longitudinal treatment history, safety constraints from the governance module, or some combination thereof. The pharmacology aggregation module 260 may generate the personalized treatment plan.

The system may further provide the personalized treatment to a clinician client device, e.g., for presentation on the interface, to obtain approval prior to execution with the patient.

### Example Machine-Learning Models

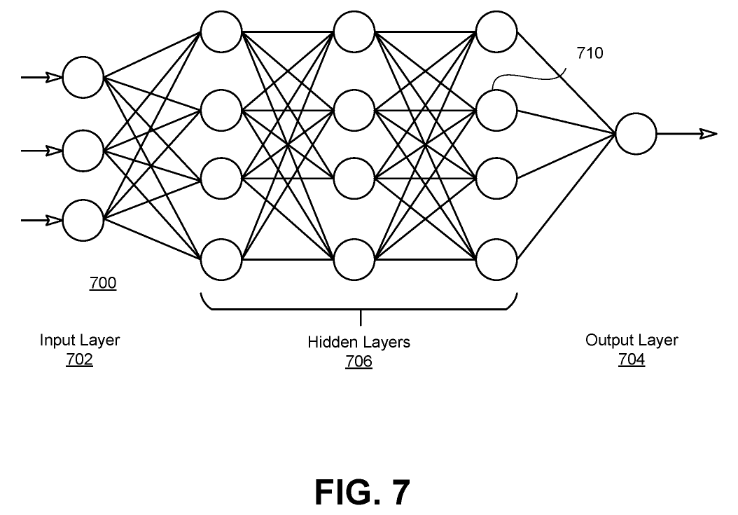

FIG. 7 shows a block diagram of a neural network model, according to one or more embodiments. The neural network 700 may receive an input and generate an output. The input may be the multimodal feature vector derived from patient data (text, audio, video, physiological signals), and the output may be predictions of current state variables or proposals for therapeutic interventions. The network may include convolutional layers for processing visual data, transformer layers for text semantics, and recurrent sequences for temporal correlation across sessions.

The order and number of layers may vary by modality. Convolutional layers may be used for facial-expression detection; recurrent or transformer layers may model conversation dynamics and affective trajectories. Kernel sizes and attention heads may differ for processing fine-grained emotional cues versus longer temporal dependencies.

Training may include forward propagation and back-propagation across nodes associated with functions such as convolution, pooling, attention weighting, and activation (e.g., ReLU, tanh). Each node’s operation reflects transformations relevant to emotion recognition, language understanding, or physiological signal interpretation.

Training of a machine learning model may include iterative forward and backward passes using mental-health session data. For instance, a computing device may receive a training set of past multimodal sessions labeled with therapeutic outcomes. For each training sample, predicted emotional state or therapy effectiveness is generated and compared with clinician-verified labels. The system adjusts network weights through stochastic gradient descent to minimize the chosen loss function.

Each function in the neural network may include coefficients adjusted during training. Activation functions (ReLU, sigmoid, tanh) control nonlinear mapping of extracted features representing voice prosody or text sentiment. Performance is evaluated by comparing predictions (e.g., mood state change, engagement score) to ground-truth outcomes measured post-therapy.

Multiple training rounds may be performed until convergence, after which the trained model infers patient states or generates interventions during live sessions. The trained model predicts risk, engagement decay, or therapeutic response probability for decision support in ongoing care.

In some embodiments, the system periodically retrains the model on newly collected session data to improve accuracy and adapt to patient population drift. Retraining may occur as part of a continuous-learning cycle in which each verified intervention outcome updates the training corpus and fine-tunes the generative and inference models for better personalization and safety alignment.

In some embodiments, model distillation may transfer knowledge from large generative or multimodal reasoning models to smaller local models. For instance, a remote transformer-based generative model (teacher) may generate therapeutic recommendations, and a simplified local model (student) may learn from those outputs to operate on edge devices with reduced latency and footprint.

Feature-based distillation may align embeddings between the teacher model’s multimodal transformer and a student model, preserving latent affective and linguistic features while reducing computational cost. Hybrid approaches may combine response- and feature-level distillation to maintain therapeutic interpretability and efficiency.

This distillation process allows clinical AI deployments (e.g., on patient mobile apps or clinician dashboards) to achieve inference consistency while meeting regulatory and safety constraints, ensuring lower latency, privacy protection, and compliant operation in healthcare contexts.

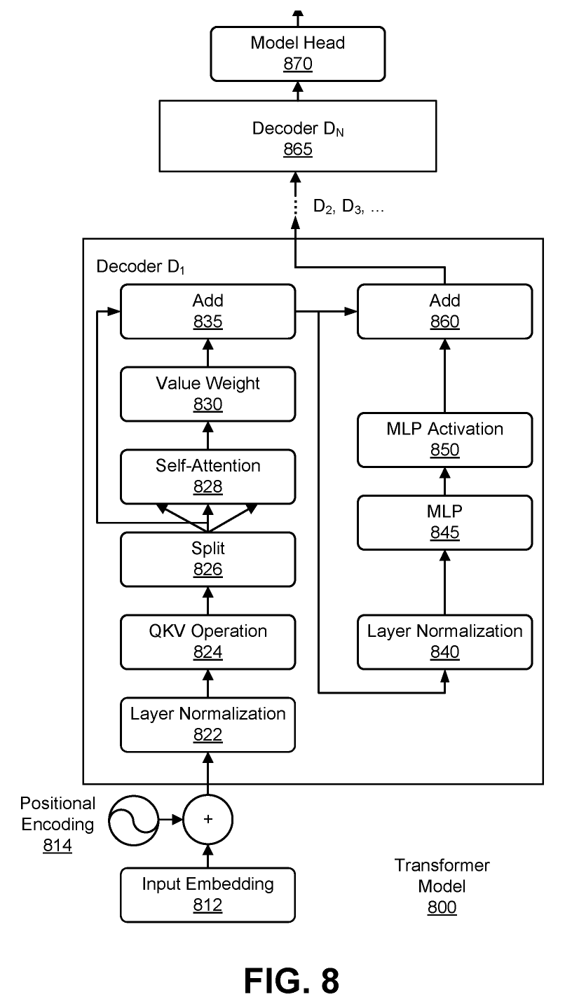

FIG. 8 is a conceptual diagram of functional blocks of a transformer-based neural network model 800, in accordance with some embodiments. For simplicity, the transformer-based neural network model 800 is referred to as a transformer model 800. The transformer model is an example of a machine-learning model discussed in this disclosure. An actual transformer model 810 may be a large language model that involves numerous neurons, such as a large number of decoders and parameters. The structure illustrated in FIG. 8 is part of a decoder for generating token attention. In a language-processing task related to therapeutic reasoning and intervention generation, the input may take the form of a sequence of words representing a structured prompt encoding multimodal state features and graph context. Each token represents a respective embedding in a latent space. Based on the input tokens, the transformer model 800 repeatedly generates a sequence of output tokens in an autoregressive manner that correspond to candidate therapeutic actions or interpretive rationales for clinical prompts.

In some embodiments, a transformer model 800 includes a set of N decoders, D1, D2, … DN. Each decoder receives input representations and generates output representations. For example, the first decoder D1 generates intermediate embeddings contextualized for patient psychological state variables and multimodal cues. Each subsequent decoder refines these embeddings using prior decoder outputs and the state-graph context until a final therapeutic recommendation vector is produced. Some decoders may correspond to analyzing data dimensions that model text, audio, video, and physiological indicators. These multimodal streams are used to perform feature integration and therapeutic inference, enabling the model to reason across linguistic, affective, and behavioral data.

The transformer model 800 may include a model head block 880 that receives the set of output representations from the final decoder DN and generates an output token as the output for the current iteration. This output may represent natural-language therapeutic guidance, clinician-facing documentation text, or intervention rationale according to safety and regulatory policies managed by the analytics system.

As shown in FIG. 8, a decoder in the transformer model 800 includes a first layer-normalization block 822, a query-key-value (QKV) operation block 824, a split block 826, a self-attention block 828, a value-weight block 830, a first add block 835, a second layer-normalization block 840, multi-layer perceptron (MLP) block 845, an MLP activation block 850, and a second add block 860. The operations in the first decoder D1 are exemplary; subsequent decoders may include similar operations. These layers allow the model to attend dynamically to relevant features in multimodal inputs and to latent state-graph variables describing the patient’s historical therapeutic context.

FIG. 8 illustrates a flow for the attention mechanism of a transformer model 800. The transformer model 800 receives an input sequence such as encoded state-graph data and multimodal embeddings collected from patient and clinician-facing interfaces. Each symbol is converted into a token that takes the form of an embedding vector. The sequence of symbols is represented as a matrix of embedding vectors, each embedding arranged in a row of the matrix. The layer-normalization block 822 receives the matrix and normalizes its values to stabilize input variance across sessions and modalities.

During training, the transformer model 800 may be trained in an autoregressive manner using masked label prediction. The input may be a therapeutic prompt sequence encoding prior session data and partially masked outcome labels. To simulate intervention prediction, the system applies masking where unknown intervention types or outcomes are hidden. The decoder attends only to previously observed state nodes and validated interventions while predicting masked positions corresponding to outcome nodes. The objective minimizes prediction error between masked positions and true therapeutic identifiers, enabling the transformer to model long-range dependencies and infer causal relationships between patient states and therapy outcomes.

The QKV operation block 824 receives the normalized dataset and performs projections to generate query, key, and value matrices. The operation applies learned weights to align representations with contextual signals derived from the longitudinal clinical memory graph (LCMG). The QKV operation models relationships among psychological variables, intervention history, and multimodal affect markers to produce attention distributions guiding therapeutic reasoning and content generation.

The split block 826 splits the QKV output into query, key, and value matrices. The self-attention block 828 uses these matrices to generate an attention matrix, applying softmax scaling. The softmax converts logit scores into attention probabilities indicating relevance between patient states and proposed interventions. This attention function allows the transformer model 800 to associate multimodal patterns with therapy outcomes and prioritize nodes with higher causal relevance within the longitudinal graph.

The value-weight block 830 receives the attention-score data to generate an attention dataset representing weighted combinations of value vectors. The results are concatenated in the add block 835 and further normalized by 840. These operations refine the interpretive context and produce latent embeddings that encode therapeutic rationale, patient progress indicators, and confidence metrics for graph updates.

Each decoder may include one or more MLP blocks 845 and MLP activation blocks 850 configured with nonlinear activation functions. The activation functions introduce non-linearity and support mapping of complex psychological transitions. Common activations may include ReLU, tanh, sigmoid, or GeLU. The MLP layers perform feature extraction across multimodal signals, generate compact context embeddings, and select token sequences for subsequent decoding related to therapy planning. Outputs are concatenated by 860 to complete the cross-modal fusion pipeline.

The output of the first decoder D1 is passed to subsequent decoders until final output data are generated. Each decoder may operate with different trained parameters focused on progressively refined aspects of patient mental-state modeling or intervention inference. The model head block 880 receives the final output from DN and determines an output token that forms natural-language recommendations or log entries. A softmax operation performed at the LM head selects the next token, resulting in governed therapeutic language or clinician summary text integrated into longitudinal documentation of patient progress.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A computer-implemented method for detecting cross-modal affective divergence in a psychiatric assessment context, comprising:
accessing multimodal biometric data from a patient captured by one or more sensors;
encoding, by a plurality of modality-specific encoder neural networks, each modality of the multimodal biometric data into a modality-specific affective state representation;
projecting, by learned modality-specific alignment projections trained with a contrastive alignment loss, each modality-specific affective state representation into a shared affective latent space;
computing a pairwise divergence score between each pair of aligned modality representations in the shared affective latent space;
computing a cross-modal affective divergence score representing the clinical significance of detected divergence based on the pairwise divergence scores;
computing, based at least in part on the cross-modal affective divergence score, a per-modality trust weight indicating estimated reliability of each modality as an indicator of the patient’s affective state;
generating a trust-weighted fused affective state estimate by combining the aligned modality representations according to the per-modality trust weights; and
generating a personalized treatment plan based on the trust-weighted fused affective state.

2. The computer-implemented method of claim 1, the multimodal biometric data comprising at least two selected from the group of: text data, voice audio data, facial imagery data, and physiological sensor data.

3. The computer-implemented method of claim 1, wherein each modality-specific encoder is independently trained.

4. The computer-implemented method of claim 1, wherein the contrastive alignment loss is configured to produce aligned representations that cluster together when modalities convey concordant affective states and separate when modalities convey discordant affective states.

5. The computer-implemented method of claim 1, wherein computing the pairwise divergence score comprises computing a complement of a cosine similarity between the pair of aligned modality representations in the shared latent space.

6. The computer-implemented method of claim 1, wherein computing the pairwise divergence score comprises:
generating a divergence matrix comprising all pairwise divergence scores arranged in the divergence matrix.

7. The computer-implemented method of claim 1, further comprising:
computing a verbal-non-verbal divergence score between a textual modality each non-textual modality; and
identifying the maximum verbal-non-verbal divergence score,
wherein computing the cross-modal affective divergence score is further based on the maximum verbal-non-verbal divergence score.

8. The computer-implemented method of claim 1, further comprising:
computing a somatic coherence score representing similarity across non-verbal modalities,
wherein computing the cross-modal affective divergence score is further based on the somatic coherence score.

9. The computer-implemented method of claim 8, wherein computing the somatic coherence score comprises:
computing a cosine similarity between each pair of non-verbal modalities; and
computing an average of the cosine similarities between the pairs of non-verbal modalities as the somatic coherence score.

10. The computer-implemented method of claim 1, further comprising:
applying a divergence characterization model to the pairwise divergence scores and the cross-modal affective divergence score to output a classification of divergence pattern exhibited in the multimodal data,
wherein generating the personalized treatment plan is further based on the classification of the divergence pattern.

11. A non-transitory computer-readable storage medium storing instructions for detecting cross-modal affective divergence in a psychiatric assessment context, the instructions that, when executed, cause one or more processors to perform operations comprising:
accessing multimodal biometric data from a patient captured by one or more sensors;
encoding, by a plurality of modality-specific encoder neural networks, each modality of the multimodal biometric data into a modality-specific affective state representation;
projecting, by learned modality-specific alignment projections trained with a contrastive alignment loss, each modality-specific affective state representation into a shared affective latent space;
computing a pairwise divergence score between each pair of aligned modality representations in the shared affective latent space;
computing a cross-modal affective divergence score representing the clinical significance of detected divergence based on the pairwise divergence scores;
computing, based at least in part on the cross-modal affective divergence score, a per-modality trust weight indicating estimated reliability of each modality as an indicator of the patient’s affective state;
generating a trust-weighted fused affective state estimate by combining the aligned modality representations according to the per-modality trust weights; and
generating a personalized treatment plan based on the trust-weighted fused affective state.

12. The non-transitory computer-readable storage medium of claim 11, the multimodal biometric data comprising at least two selected from the group of: text data, voice audio data, facial imagery data, and physiological sensor data.

13. The non-transitory computer-readable storage medium of claim 11, wherein each modality-specific encoder is independently trained.

14. The non-transitory computer-readable storage medium of claim 11, wherein the contrastive alignment loss is configured to produce aligned representations that cluster together when modalities convey concordant affective states and separate when modalities convey discordant affective states.

15. The non-transitory computer-readable storage medium of claim 11, wherein computing the pairwise divergence score comprises computing a complement of a cosine similarity between the pair of aligned modality representations in the shared latent space.

16. The non-transitory computer-readable storage medium of claim 11, wherein computing the pairwise divergence score comprises:
generating a divergence matrix comprising all pairwise divergence scores arranged in the divergence matrix.

17. The non-transitory computer-readable storage medium of claim 11, the operations further comprising:
computing a verbal-non-verbal divergence score between a textual modality each non-textual modality; and
identifying the maximum verbal-non-verbal divergence score,
wherein computing the cross-modal affective divergence score is further based on the maximum verbal-non-verbal divergence score.

18. The non-transitory computer-readable storage medium of claim 11, the operations further comprising:
computing a somatic coherence score representing similarity across non-verbal modalities,
wherein computing the cross-modal affective divergence score is further based on the somatic coherence score.

19. The non-transitory computer-readable storage medium of claim 18, wherein computing the somatic coherence score comprises:
computing a cosine similarity between each pair of non-verbal modalities; and
computing an average of the cosine similarities between the pairs of non-verbal modalities as the somatic coherence score.

20. The non-transitory computer-readable storage medium of claim 11, the operations further comprising:
applying a divergence characterization model to the pairwise divergence scores and the cross-modal affective divergence score to output a classification of divergence pattern exhibited in the multimodal data,
wherein generating the personalized treatment plan is further based on the classification of the divergence pattern.

21. A system for detecting cross-modal affective divergence in a psychiatric assessment context, the system comprising:
one or more processors; and
a non-transitory computer-readable storage medium storing instructions that, when executed, cause the one or more processors to perform operations comprising:
accessing multimodal biometric data from a patient captured by one or more sensors;
encoding, by a plurality of modality-specific encoder neural networks, each modality of the multimodal biometric data into a modality-specific affective state representation;
projecting, by learned modality-specific alignment projections trained with a contrastive alignment loss, each modality-specific affective state representation into a shared affective latent space;
computing a pairwise divergence score between each pair of aligned modality representations in the shared affective latent space;
computing a cross-modal affective divergence score representing the clinical significance of detected divergence based on the pairwise divergence scores;
computing, based at least in part on the cross-modal affective divergence score, a per-modality trust weight indicating estimated reliability of each modality as an indicator of the patient’s affective state;
generating a trust-weighted fused affective state estimate by combining the aligned modality representations according to the per-modality trust weights; and
generating a personalized treatment plan based on the trust-weighted fused affective state.
